(19) United States
(12) Patent Application Publication
(10) Pub. No.: US 2010/0160774 A1
(43) Pub. Date: Jun. 24, 2010

(54) Method and apparatus for non-invasive cancerous tissue diagnosis and tomography using terahertz imaging

(76) Inventors: Ali Dabiri, Brier, WA (US); Manouchehr Motamedi, Sugar Land, TX (US)

Correspondence Address: Manouchehr Motamedi, 1002 Ancrum Hill Lane, Sugar Land, TX, 77479, US

(21) Appl. No.: 12/655,593

(22) Filed: Jan. 4, 2010

Related U.S. Application Data
(63) Continuation-in-part of application No. 10/801,922, filed on Mar. 17, 2004, now Pat. No. 7,643,869.

(51) Int. Cl. A61B 6/00

(52) U.S. Cl. 600/425

(57) ABSTRACT

The primary objective of the present method and apparatus is to provide a transportable diagnosis system to examine the conditions of a human tissue. The method uses the most advanced Terahertz imaging system to detect and analyze cancerous tissues. The method has objective to detect and analyze cancerous tissues by comparing a plurality of spectrum resolved images of suspected tissue without applying harmful agents into the tissue to facilitate interaction with illumination sources. The method employs non-evasive, real time terahertz imaging systems and techniques to diagnose tissue for detecting the presence of cancer. A map showing, which tissue is healthy and which is cancerous can aid in the accurate removal of cancerous tissue.

### FIELD OF THE DISCLOSED METHOD AND APPARATUS

This method and apparatus is related to non-invasively diagnosing cancerous tissue, using terahertz systems and more specifically to tomography of the tissue using terahertz imaging.

### BACKGROUND

The tissue diagnosis of cancer is routinely performed by standard surgical histo-pathological analysis, which involves preparation of tissue and then viewing under the microscope. This method is painful for patients, time consuming, and its results can take up to a few days to be ready. Direct visualization is also used, but is weakened by subjective decision. Currently, a frozen section diagnostic method is employed for operating room situations, where there is a need for immediate results. This technique can take up to 30 minutes. There are major difficulties with this method, primarily due to inaccurate diagnosis and a large amount of artifact in the tissue preparation. One of the most difficult issues involves finding a location having an appropriate surgical margin.

A number of systems have been developed in the past twenty years to perform biopsy on tissue by optical techniques. The early nineteen-eighties saw the development of simple systems which would irradiate either UV, visible, or infrared lights onto the tissue, and attempt to characterize the resulting reflectance or fluorescence emission spectra. This type of illumination was not always specific. Furthermore, high levels of tissue autofluorescence were observed. Therefore, there was a need to develop specific tumor markers such as Homoproto Porphorin Derivative (HPD). These substances were accordingly developed. HPDs are injected into tissue before laser illumination. After a suitable time, laser interrogation is performed. Problems of toxicity, patient safety, and convenience have prevented the widespread use of these photodynamic agents.

Later attempts to use laser induced fluorescence as a diagnostic tool resulted in the development of simple single fiber systems. Such systems illuminate the suspect areas of tissue with laser light at a focal point where a single fiber relays the tissue fluorescence. Actual spectral differences were found to exist between normal tissue and abnormal tissue. These studies have led to an array of spectral analysis technique, but they have all been limited to three or four spectral lines corresponding to reflectance of fluorescence signature peaks. The exact reason for the differences in normal versus cancerous tissue are not understood, but might be related to the three-dimensional structure or differences in biochemical makeup. The ability to exploit these differences can be used as a diagnostic tool.

Although these systems are relatively simple to use and can be adapted to existing endoscopic and colonoscopic instruments for measurements, they have three fundamental limitations:
1. The techniques can easily miss a small tumor since single fiber illumination area is extremely small.
2. The techniques cannot provide information on surgical margins during operative procedure due to lack of imaging capabilities.
3. Most techniques require the application of photodynamic agents.

Mooradian et al., in U.S. Pat. No. 5,782,770 have proposed a technique for diagnosing tissue via hyperspectral imaging. In this technique, the spectral content of the image can be analyzed on a pixel-by-pixel basis to determine the presence of certain matter. Although this technique operates in real time and is non-invasive, it provides information only from the surface of the tissue. A real time three-dimensional tomography is needed to fully differentiate normal tissue from abnormal tissue.

Accordingly, a real time, non-invasive method is needed to rapidly diagnose the tissue, reduce the uncertainty of tissue diagnosis, and provide an actual image to identify the exact surgical margins during operative procedures.

### BRIEF SUMMARY OF THE DISCLOSED METHOD AND APPARATUS

The primary objective of the present method and apparatus is to provide new systems and methods for the diagnosis of tissue conditions. It is also the objective of the present method and apparatus to provide systems and methods for detecting and analyzing cancerous tissues by comparing a plurality of spectrum resolved images of suspected tissue.

The present method and apparatus employs non-evasive, real time terahertz imaging systems and techniques to diagnose tissue for detecting the presence of cancer. The present method and apparatus can be distinguished from similar techniques in that a terahertz image contains detailed spectral information which can be analyzed for spectral signature characteristics not found in auto-fluorescence and similar emission mechanisms.

Sensing with terahertz segment of spectrum has several significant advantages over sensing at other sides of spectrum. Terahertz radiation is completely unionized with photon energies more than six orders of magnitude less than soft x-rays. Most terahertz applications require less than one microwatt of power makes terahertz radiation completely safe for use by humans or on human subjects.

Terahertz waves are a segment of electromagnetic waves. Terahertz waves are bounded between millimeter waves (less than 1×10¹¹ Hz) and photonics waves (greater than 1×10¹³). The electromagnetic frequencies lower than terahertz band are covering mm waves (microwaves), while the electromagnetic frequencies higher than terahertz band are covering near infrared through visible spectrum.

Terahertz wave band can be used for time domain and frequency domain imaging. The present applications of terahertz are spectroscopy in atmospheric science and in astronomy, imaging for burn diagnostics, tomography, biomedical, medical diagnostics, screening for weapon, explosives, biohazard, and finally imaging of concealed objects.

Existing water in living tissue limits the penetration depth of terahertz energy to a few millimeters, which is just sufficient for the diagnosis of cancerous tissue.

Besides detecting the presence of cancer, the present method and apparatus is also valuable to locate the extent of the spread of cancerous tissue as well as the progression of the cancer. A map showing, which tissue is healthy and which is cancerous, can aid in the accurate removal of cancerous tissue. However, systems employing single point detection do not show the extent of the affected tissue region. But when using terahertz systems such as disclosed herein, the combination of the spatial resolution and the high spectral resolution of a terahertz imaging system can be utilized to detect cancerous tissues.

The present method and apparatus does not require introduction of harmful agents into the tissue.

The present method and apparatus gathers data in three spatial dimensions. Initially, a spot is selected on the tissue and a line image is constructed through the depth of the tissue. The depth of the tissue is binned according to the desired spatial resolution and signal-to-noise ratio. Then, the incident beam will be scanned in a horizontal direction alongside of the tissue. Successive line images along the depth of the tissue will be collected. Finally, the beam will be scanned in the orthogonal direction to start a new horizontal scan. The same information will be gathered successively over the entire tissue. A tomography will then be constructed from the gathered overall data.

A better understanding could be achieved with reference to Detailed Description of the disclosed method and apparatus and with reference to the drawing. The description represents a particular case to realize the disclosed method and apparatus and is not intended to define the invention, but merely to provide adequate support for the claims appended hereto. Accordingly, the invention is defined solely by the claims to the invention appended hereto.

### BRIEF DESCRIPTION OF THE DRAWINGS

FIG. 1 is a simplified diagram of the THz scanning the tissue using standard methods of both time domain (TD) and frequency domain (FD) tomography.

### DETAILED DESCRIPTION

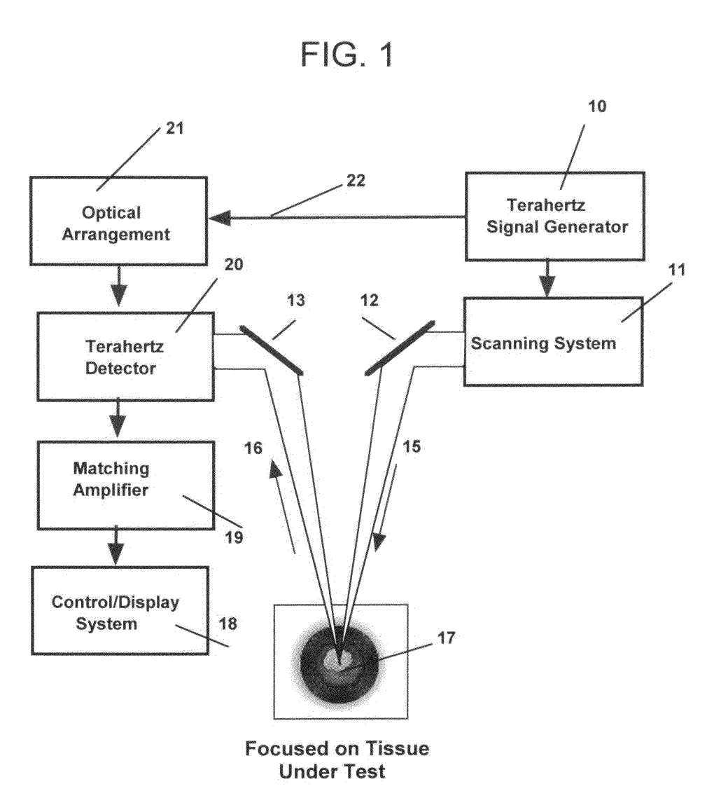

The FIG. 1 is a simplified diagram of the THz scanning of tissue using standard methods of both time domain (TD) and frequency domain (FD) tomography. A tailored source of terahertz signal generator 10 is fed to the scanning system 11. Scanning system 11 includes positioner and collimation and focusing optics where the focused THz rays are directed by mirror 12 on the tissue and positioned at specific location 17. The THz rays are focused at one point, stays at that point until travel inside the tissue creating data of depth layers, and then it scans to the next point until the required area is 3-D scanned. Based on the doctor's requirement positioner can select different location for collecting similar data. The reflections, of THz rays from the tissue are redirected to Terahertz detector 20 via mirror 13. The terahertz signal generator 10 generates both pulsed Terahertz and Terahertz frequency sweeps sources for TD and FD methods respectively. These sources are transmitted to optical arrangement system 21 through line 22. Optical Arrangement system 21 creates variable optical delay lines and local sweep oscillators to feed to detector 20 for TD and FD methods respectively. The Terahertz detector 20 extracts tissue data using variable delay lines in the case of the TD method and local sweep oscillators and mixer in the case of FD method and transfer data to matching amplifier system 19 and then the collected data will arrive in control/display system 18 where the overall tissue data is analyzed and the results is sent electronically to the patient's medical report.

The tissue image is comprised of a plurality of horizontal bands, each band being adjacent to another, with equal bandwidths and comprised of a plurality of pixels where each pixel being adjacent to another. The images with a calibrated reference are stored in a memory, indicating regions of coincidence and regions of non-coincidence, and combining the images at different layers to obtain the tomography of the tissue.

We claim:

1. An apparatus for diagnosis of tissue, comprising:
a) an electromagnetic source;
b) a scanning system coupled to the electromagnetic source;
c) a scanning system to scan the tissue in two dimensions
d) an optical arrangement system to generate reference sources for detector
e) a detector
f) a matching amplifier coupled to the detector;
g) a control/display system to receive data from matching amplifier to interpret the test results.

2. The apparatus of claim 1, wherein the electromagnetic source is a Terahertz signal generator.

3. The apparatus of claim 1, wherein the scanning system includes collimation, focusing, and beam positioner systems.

4. The apparatus of claim 1, wherein the optical arrangement includes variable optical delay lines and Terahertz sweep generators,

5. The apparatus of claim 1, wherein the scanner and display system are inside a transportable system together with the other elements recited in claim 1.

6. A method for diagnosis of tissue, comprising:
a) generate and tailor Terahertz signal;
b) split Terahertz signal to scanning system and optical arrangement;
c) inserting into the optical arrangement a Terahertz detector;
d) inserting the output of the Terahertz detector to a matching amplifier
e) Inserting the output of the matching amplifier to a control and display system
f) illuminating the tissue with a focused scanning Terahertz beam; and
g) redirecting the reflection of the tissue into detector.

7. The method of claim 6, further comprising using a scanning system dynamically synchronized to the scanned terahertz signals that are illuminating the tissue.

8. The method of claim 6, wherein the Terahertz signal generator covers terahertz bandwidth from 200-10000 GHz.

9. The method of claim 7, further comprising setting up a terahertz detector to detect either the difference signal from the reflected terahertz signal from the tissue and the reference signal split from the Terahertz signal generator, or mixer output when inserting local sweep generator and reflected terahertz signal from the tissue to the two inputs of the mixer.

10. The method of claim 6, further comprising using matching amplifiers to improve the detected signals.

11. The method of claim 6, further comprising forming an image from the reflected pulses at each layer perpendicular to the tissue surface.

12. The method of claim 6, further comprising:
a. comparing the images with a calibrated reference stored in memory;
b. combining the images at different layers to obtain the tomography of the tissue;
c. indicating regions of coincidence and regions of non-coincidence; and
d. showing the result in control and display system.

13. The method of claim 6 wherein the detector acts as a convolver to detect the reflected terahertz signals from the tissue, which arrive at detector, synchronized to its split reference beam.

14. The method of claim 6 wherein the detector acts as a mixer to detect the difference signal between reflected terahertz signals from the tissue and the local sweep oscillator arrives from optical arrangement.

15. The method of claim 11, further providing a three dimensional image of the tissue in real time which includes compositional information about the tissue.

16. The method of claim 12 wherein the image is comprised of a plurality of horizontal bands, each band being adjacent to another, with equal bandwidths

17. The method of claim 15, further including comparing the images with a calibrated reference stored in memory, indicating regions of coincidence and regions of non-coincidence, and combining the images at different layers to obtain the tomography of the tissue.
